(19) United States
(12) Patent Application Publication
(10) Pub. No.: US 2022/0387168 A1
(43) Pub. Date: Dec. 8, 2022

(54) CORNEAL TISSUE

(71) Applicant: THE UNIVERSITY OF NOTTINGHAM, Nottinghamshire (GB)

(72) Inventors: Laura Sidney, Nottingham (GB); Andrew Hopkinson, Nottingham (GB); Owen McIntosh, Nottingham (GB)

(21) Appl. No.: 17/776,990

(22) PCT Filed: Nov. 13, 2020
(86) PCT No.: PCT/GB2020/052906

§ 371 Date: May 13, 2022

(30) Foreign Application Priority Data
Nov. 13, 2019 (GB) 1916506.7

(51) Int. Cl. A61F 2/14; A61L 27/36; C12N 5/079

(52) U.S. Cl. CPC A61F 2/142; A61L 27/3604; A61L 27/3666; A61L 27/3691; C12N 5/0621; A61L 2430/16

(57) ABSTRACT

The invention provides an isolated dehydrated corneal tissue, comprising a full thickness corneal stroma, and substantially all, or all, of the Bowman's membrane, wherein the stroma contains cellular material. Also provided is a method to product the tissue and uses of the tissue.

This invention relates to corneal tissue, in particular to dehydrated corneal tissue, more specifically dehydrated corneal tissue that has been denuded of its outer cellular layers but retains cellular material in the stroma. The invention also relates to a method of preparing dehydrated and denuded corneal tissue and uses of the corneal tissue, and methods of treatment involving the dehydrated corneal tissue and solutions for rehydrating a dehydrated corneal tissue.

There are over 10 million untreated corneal-blind patients globally, with approximately 1.5 million new cases diagnosed annually. Corneal transplantation can restore vision with a 5-year graft survival rate of 74%. However, 1 in 6 full thickness transplants suffer rejection due to variable quality in epithelial or endothelial health. In these cases, the patient may lose their sight and even their eye.

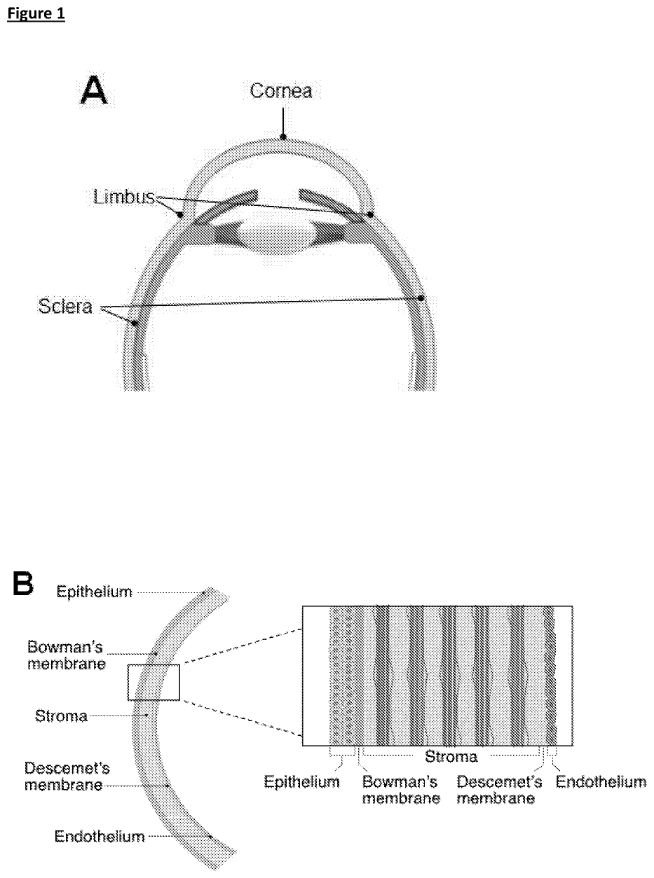

The cornea is the transparent surface of the eye (see FIG. 1), and comprises three major cellular layers, i.e. the epithelium, the stroma and the endothelium. The epithelium, the outermost cellular layer of the cornea, is separated from the stroma by the Bowman's membrane, and the endothelium, the innermost cellular layer of the cornea, is separated from the stroma by the Descemet's membrane. When corneal transplants are rejected by a host's immune system, this usually occurs due to complications involving the epithelial and/or endothelial layer(s). Donor corneas are usually stored in an eye bank, in organ culture media, under specialised environmental conditions, for two weeks to four weeks before being used in surgery. This means that they cannot easily be shipped globally, and usually have to be used locally. They are also expensive to store.

Currently, there is a global shortage of donated corneas suitable for transplantation. One of the biggest limitations to corneal transplantations is access to quality donor tissue due to inadequate eye donation services and infrastructure in developing countries. This is compounded by the fact that there are no long-term storage solutions for effectively preserving spare donor corneas collected in countries with a surplus. Eye banking infrastructure requires large amounts of local investment and labour to put it in place.

There is a need to increase access to corneal tissue. One way to provide access would be to develop preservation techniques to increase corneal storage times and allow for global shipping at ambient temperature. Such preservation would also allow storage on hospital shelves, for use in emergencies, where waiting for donor corneas is not possible. The present invention provides an improved isolated natural corneal tissue which can be used for transplantation.

According to a first aspect, the invention provides an isolated dehydrated corneal tissue, comprising a full thickness corneal stroma, and substantially all, or all, of the Bowman's membrane.

The dehydrated corneal tissue may also comprise some or substantially all, or all, of the Descemet's membrane.

Preferably the dehydrated corneal tissue does not comprise any of the epithelium or endothelium (the epithelium and endothelium are naturally found on the cornea). As the corneal tissue according to the invention does not possess an epithelium or an endothelium, it is less likely to trigger an immune response in a recipient, and thus the corneal transplant is less likely to be rejected.

The dehydrated corneal tissue may consist of all or substantially all of the corneal stroma, and substantially all or all of the Bowman's membrane.

The dehydrated corneal tissue may consist of all or substantially all of the corneal stroma, substantially all, or all, of the Bowman's membrane and substantially all, or all, of the Descemet's membrane.

In the dehydrated corneal tissue of the invention, preferably the stroma contains cellular material. Preferably the stroma retains some or all of the cells from the original tissue in the dehydrated tissue. Preferably in the stroma of the dehydrated corneal tissue of the invention at least some of the corneal keratocytes are retained. Whilst the retained cells may no longer be alive in the dehydrated tissue, at least some of the cellular components may be retained, for example membrane components and the DNA. Using microscopy the shape of the retained cells, in particular the cell membrane and nucleus, may be observed in the dehydrated tissue. This is in contrast to other known dehydrated corneal tissues, which are actively decellularised or are acellular, that is the tissue has been treated to remove any cellular material prior to dehydration. In the dehydrated corneal tissue of the invention the stroma is not acellular and/or has not been decellularised.

The dehydrated corneal tissue of the invention may have an osmotic agent, such as a glucose polymer, for example dextran, on the surface,

The dehydrated corneal tissue of the invention may include a drying protectant, such as raffinose or a saccharide, for example trehalose, sucrose, stachyose and/or verbascose, within or on the tissue

The isolated dehydrated corneal tissue of the invention may comprise substantially the entire diameter, or the entire diameter, of a donor's cornea. The corneal tissue may not comprise the limbus or the sclera. The corneal tissue may further comprise all or substantially all, of a donor's limbus. The corneal tissue may further comprise all or substantially all of a donor's sclera. The term “limbus” can refer to the region adjacent to and surrounding the circumference of a cornea. Thus, the “limbus” may be the border between the transparent cornea and the white sclera. The term “sclera” can refer to a region adjacent to the limbus. The term “sclera” be the white outer layer of the eyeball which surrounds the circumference of the limbus and the cornea.

The corneal tissue according to the invention may not be a lenticule. The term “lenticule” can refer to corneal tissue that is less than the full thickness of the corneal stroma.

The corneal tissue according to the invention may or may not be a corneal button. The term “corneal button” can refer to corneal tissue that comprises corneal stroma, preferably the full thickness of the corneal stroma, which comprises less than the full diameter of the donor's cornea.

Current methods of preparing donor corneas for transplantation results in corneal tissue that comprises living endothelial and/or epithelial cells as well as living keratocytes within the stromal layer (stroma) from the donor. Corneal tissue according to the invention may not comprise any viable/living cells or live cellular layers from the donor. In an embodiment, the corneal tissue according to the invention does not comprise any viable/living cells or live cellular layers from the donor and can be used as an alternative to traditional, live donor corneas.

Dehydrated corneal tissue according of the invention also has the advantage that it can be stored for many weeks, months and indeed years. Preferably the dehydrated corneal tissue of the invention can be viably stored for at least two years. This is significantly longer than the maximum period of four weeks that current live donor corneal tissue can be stored. The corneal tissue of the invention also has the advantage that it can be stored dry at room temperature. Room temperature may be between about 4° C. and 60° C., preferably between about 15° C. and 37° C., most preferably at about 25° C. The increased storage time and room temperature storage of the corneal tissue according to the invention means it can be readily transported all over the world for use in corneal transplants.

Prior to transplantation into the recipient/subject, the dehydrated corneal tissue of the invention is rehydrated. Once transplanted, the rehydrated corneal tissue may initially function as a scaffold for the recipient's cells. These cells may be stem cells. The cells may differentiate to form an epithelium on the transplanted corneal tissue and the host's stromal cells may migrate to populate the transplanted stroma.

Unlike synthetic keratoprotheses, the corneal tissue according to the invention retains the ultrastructure and biological signals of the native tissue. This is advantageous because it aids integration of the donor corneal tissue with the host (recipient's) tissue, which improves its ability to function as a cornea once transplanted into a recipient.

In a preferred embodiment of the invention the dehydrated corneal tissue is not prepared by freeze-drying.

In a preferred embodiment of the invention the dehydrated corneal tissue has not been lathed. In other known methods of preparing corneal tissue for transplantation outer tissue layers are removed by lathing the tissue.

According to a second aspect of the invention, there is provided a method of producing dehydrated corneal tissue, the method comprising:
a) providing corneal tissue that has been obtained from a donor;
b) suspending the corneal tissue in a solution comprising an osmotic agent;
c) agitating the solution in which the corneal tissue is suspended in order to remove the endothelial cells and epithelial cells; and
d) vacuum-drying the corneal tissue from c) in order to produce a dehydrated corneal tissue.

The method according to the invention is advantageous because it is scalable and cost effective. As the resulting tissue does not contain living cells it also means that tissue harvested from a deceased individual (donor) more than 24 hours after their death can be used. Currently transplant methods which use live donor corneas can only use corneal tissue that was harvested up to about 48 hours after the death of the donor. After this time, the donor tissue becomes non-viable for live cell transplant purposes, and if used could cause ocular ulcers and/or ruptures.

The method according to the invention may use corneal tissue harvested up to about 96 hours after donor death, up to about 72 hours after donor death, up to about 66 hours after donor death, up to about 60 hours after donor death, up to about 54 hours after donor death, up to about 48 hours after donor death, up to about 36 hours after donor death or at least about 24 hours after the death of the donor. The method may comprise providing a cornea that has been harvested between about 24 hours and about 72 hours after the death of the donor, or between about 24 hours and about 48 hours after the death of the donor, or between about 48 hours and about 72 hours after the death of the donor. Most preferably, the method comprises providing a cornea that has been harvested between 0 hours and about 48 hours after the death of the donor. Step (a) according to the first aspect may not comprise the step of removing the cornea from the donor.

The corneal tissue provided in step (a) of the method of the invention may comprise substantially all, or all, of the epithelium, all, or substantially all, of the Bowman's membrane, all or substantially all, of the stroma, all, or substantially all, of the Descemet's membrane and all, or substantially all, of the endothelium.

After step (c) the corneal tissue may comprise substantially all, or all, of the Bowman's membrane, substantially all, or all, of the stroma and substantially all, or all, of the Descemet's membrane. Preferably after step (c) the corneal tissue does not comprise the epithelial layer (the epithelium) and/or the endothelial layer (endothelium).

In one embodiment, the corneal tissue after step (c) comprises all or substantially all of the Bowman's membrane and all or substantially all of the stroma. After step (c) the corneal tissue may also comprise all or substantially all of the Descemet's membrane.

In one embodiment, the corneal tissue after step (c) consists of all or substantially all of the Bowman's membrane and all or substantially all of the stroma. In another embodiment, the corneal tissue after step (c) consists of all or substantially all of the Bowman's membrane, all or substantially all of the stroma and all or substantially all of the Descemet's membrane.

Preferably, the Descemet's membrane and/or the Bowman's membrane of the donor are not intentionally removed from the corneal tissue.

Preferably in step c) the keratocytes in the stroma are retained. Preferably the stroma is not decellularised.

The corneal tissue may comprise substantially the entire diameter of the donor's cornea. The corneal tissue may not be a lenticule. Preferably, the stroma of the corneal tissue is of full thickness.

The corneal tissue may further comprise some or substantially all of the limbus of the donor. The corneal tissue may further comprise some or substantially all of the sclera of the donor. The corneal tissue may not comprise the limbus or the sclera.

Removal of a cornea from the eye of a donor usually results in the cornea swelling. Thus, the solution in which the cornea is suspended may comprise an osmotic agent that modulates (increases or decreases) the passage of water into the cornea. Preferably the osmotic agent reduces the passage of water into the cornea. Therefore, the osmotic agent may be a salt (such as NaCl, KCl or CaCl₂). Thus, the solution may be saline. The saline may be a normal saline containing 0.9% NaCl, this is the physiological level of salt, and produces an isotonic solution with an osmolarity of 308 mOsm/l which maintains stability of cells membranes by ensuring water does not move inside or outside of cells in the solution. The osmotic agent may alternatively or additionally comprise a glucose polymer, such as dextran. Therefore, the solution in which the cornea is suspended (during step (b)) may comprise dextran. Osmotic agents, such as dextran, may reduce and/or prevent swelling of the cornea. The percentage of dextran (w/v) in the solution may be about 1% to about 10%, about 2% to about 9%, about 3% to about 8% or about 4% to about 6%. Preferably, the percentage of dextran in the solution is about 5%, most preferably about 5% dextran 70.

The solution may further comprise a drying protectant, such as raffinose. The solution may further comprise other drying protectants, such as a saccharide, for example trehalose, sucrose, stachyose and/or verbascose. Preferably, the drying protectant is raffinose. The concentration of the drying protectant, such as raffinose, may be about 100 mM or at least about 100 mM.

In the method of the invention the aim is to maintain the cells, in particular the keratocytes in the stroma, and to avoid them bursting due to hyper or hypotonic conditions. Thus the tissue is processed in a solution which maintains these cells at a physiological or neutral osmolarity. This may be achieved by using a glucose polymer, such as dextran, in a saline solution—preferably the saline solution is isotonic and prevents cells in the tissue from bursting, the addition of the glucose polymer also makes the solution iso-osmotic. In the method of the invention, once the tissue is dehydrated the cells may no longer be viable; however the cellular material is retained.

In an embodiment of the invention the solution comprises or consists essentially of 0.9% (w/v) sodium chloride (NaCl), 5% (w/v) Dextran 70, and optionally 100 mM raffinose, in water.

The agitation in step (c) is intended to remove all or substantially all the cells in the epithelial layer and all or substantially all the cells in the endothelial layer. The agitation is step is not intended to remove, burst or damage the keratocytes of the stroma. Preferably, after agitation, all, most or at least some of the keratocytes are retained in the stroma. Agitation may comprise rotation (revolving from “head to toe” or 360 degree vertical rotation), rolling and/or shaking (movement side to side or “head to toe”) of a vessel comprising the corneal tissue suspended in an appropriate solution. Rotation of the vessel may be at about 40 to about 80 revolutions per minute (rpm), at about 50 to about 70 rpm or at about 55 to about 65 rpm. Preferably, rotation is at about 60 rpm. Agitation may be performed at room temperature. Agitation, such as rotation, rolling or shaking, may be performed, at about 4° C. to about 55° C., at about 15° C. to about 25° C., at about 30° C. to about 45° C. or at about 35° C. to about 40° C. Agitation may be performed at about 37° C., alternatively agitation may be performed at room temperature, as about 20° C. Most preferably, agitation is by rotation at about 60 rpm at about 20° C., or at about 37° C. Agitation may be performed for up to about 20 minutes, for up to about 30 minutes, for up to about 60 minutes, for up to about 90 minutes, for up to about 120 minutes (2 hours), for up to about 4 hours, for up to about 6 hours, for up to about 10 hours or for up to about 12 hours. Preferably, agitation is performed for about 60 minutes. Surprisingly, agitation gently removes the epithelial layer and the endothelial layer, leaving the Bowman's membrane and preferably the Descemet's membrane intact. Removal of the epithelial layer and the endothelial layer reduces the likelihood that an immune response will be triggered in the recipient of the corneal tissue. Agitation may be achieved by rolling a vessel comprising the corneal tissue suspended in an appropriate solution.

Preferably the method of producing dehydrated corneal tissue does not include a step in which the tissue is lathed.

Preferably the method of producing dehydrated corneal tissue does not include a step in which the tissue is treated with chemicals or enzymes, such as thermolysin, which may cause irreparable damage to the structure of the cornea and/or burst cells of the endothelium and/or epithelium and leave behind DNA fragments, which may potentially induce an immune response.

The method according to the second aspect may further comprise, between step (c) and step (d), a step of storing the corneal tissue in a solution. The solution may be the solution of step (b) or another suitable solution, such a solution containing chondroitin sulfate and dextran. The solution may comprise dextran in order to reduce and/or prevent swelling of the cornea. Preferably the addition of dextran makes an iso-osmotic solution, which maintains the pressure inside and outside the cornea, and prevents water from moving into the cornea. The percentage of dextran (w/v) in the solution may be about 1% to about 10%, about 2% to about 9%, about 3% to about 8% or about 4% to about 6%. Preferably, the percentage of dextran in the solution is about 5% (w/v), most preferably about 5% (w/v) dextran 70. The method according to the second aspect may further comprise, between step (c) and step (d), a step of removing the corneal tissue from solution, such as the solution comprising an osmotic agent. The corneal tissue may be stored out of solution for up to about 2 months, for up to about 1 month, for up to about 15 days, for up to about 7 days, for up to about 3 days, for up to about 1 day, for up to about 18 hours, for up to about 12 hours, for up to about 6 hours, for up to about 3 hours, for up to about 2 hours or for up to about 1 hour.

The vacuum drying step (step (d)) may render all of the cells in the corneal tissue (including keratocytes within the corneal stroma) non-viable (i.e. not living and prevents them from carrying out any metabolic processes or dividing) but does not destroy the cell membranes.

Conventional freeze-drying requires the tissue to be frozen before it is dried. The drying phase of the freeze-drying process comprises placing the tissue in a vacuum and then drying it, for example, by applying heat to the tissue. Vacuum drying, as referred to herein, may comprise freeze-drying a tissue but without the pre-freezing step. Preferably, therefore, the vacuum-drying process does not comprise a pre-freezing step. Thus, the dehydrated corneal tissue according to the invention may not be frozen until after it has been dehydrated (i.e. after step (d)). In an embodiment the corneal tissue is never frozen.

Vacuum drying is preferred because it reduces the boiling point of water inside the cornea and increases the rate of evaporation of the water. The vacuum drying process may be performed in a dryer, such as a freeze-dryer. Preferably, the vacuum drying step is performed by vacuum evaporation. The vacuum-drying step may be performed on the corneal tissue when it is out of solution. Vacuum evaporation enables the cornea to be dried without removing the cornea from the solution in which it is suspended. This is advantageous because it reduces the number of steps required to produce a dehydrated corneal tissue. It is also advantageous because it allows the solutes of the solution to be deposited on the cornea.

The temperature at which the vacuum drying process is performed must be high enough to enable the intermolecular bonds of the water molecules in the cornea to break. The skilled person would appreciate that the temperature at which this occurs will vary depending on the partial pressure that the tissue is exposed to. Above this temperature, and under a vacuum, the heat energy also encourages the water molecules to vaporise. In one embodiment, the vacuum drying process may comprise drying at a temperature high enough to enable the intermolecular bonds of the water molecules in the cornea to break. In one embodiment, the vacuum drying process may comprise drying at a temperature that is high enough to enable the intermolecular bonds of the water molecules to break (i.e. the drying temperature does not vaporise the water molecules). The vacuum drying process may be performed at or below room temperature, such as at or below about 30° C., at or below 25° C., or at or below 20° C. The vacuum drying process may be performed at about 4° C. to about 40° C., at about 8° C. to about 40° C., at about 10° C. to about 35° C., at about 10° C. to about 30° C., at about 20° C. to about 30° C. or at about 23° C. to about 28° C. Preferably, the vacuum drying process is performed at about 25° C. The temperature recited may comprise or consist of the shelf temperature in a dryer or the atmospheric temperature in the vacuum. The temperature of the vacuum drying process may comprise or consist of heat supplied by the shelves of a dryer, such as a freeze-dryer. Thus, the heat energy used in the vacuum drying process may be transferred to the tissue by conduction, convection and/or radiation.

Vacuum drying is also preferred because placing the tissue in a vacuum (i.e. a pressure under 1 atmosphere), preferably under a pressure under 60.795 mbar (0.06 atm), encourages water to vaporise. The greater the vacuum, the greater the rate at which the water vaporises. The vacuum drying process may, therefore, be performed at about 1 μbar (0.001 mbar) to at about 60,000 μbar (60 mbar), at about 100 μbar (0.1 mbar) to at about 60,000 μbar (60 mbar), at about 1000 μbar (1 mbar) to at about 60,000 μbar (60 mbar), at about 1500 μbar (1.5 mbar) to at about 60,000 μbar (60 mbar), at about 1 μbar (0.001 mbar) to at about 1500 μbar (1.5 mbar), at about 100 μbar (0.1 mbar) to at about 5000 μbar (5 mbar), at about 300 μbar (0.3 mbar) to at about 1400 μbar (1.4 mbar), at about 600 μbar (0.6 mbar) to at about 1300 μbar (1.3 mbar), at about 800 μbar (0.8 mbar) to at about 1200 μbar (1.2 mbar), at about 900 μbar (0.9 mbar) to at about 1100 μbar (1.1 mbar). The vacuum drying process may be performed at about 990 μbar (0.99 mbar).

The vacuum drying process may be performed at above about 1.5 mbar, for example, between about 1.5 mbar and about 60 mbar.

The vacuum drying is performed at a pressure and temperature at which the tissue does not freeze.

The pressure used during the drying process may be continuous, that is a continuous pressure may be used for the full drying period. Alternatively, the pressure may change over time. For example, when the pressure is set by a vacuum pump, the vacuum may be applied only intermittently, or not continuously, such that the pressure changes intermittently, or is not always the same. For example, a designated pressure may be used for a designated time, then the vacuum may be removed or reduced the pressure may increase for a designated period of a time, and this may be repeated several times. For example, a first pressure may be used for between 1 and 10 minutes, the vacuum may then be removed or reduced, and the pressure increased, for a further 1 to 10 minutes, this cycle may be repeated between 5 and 50 times. In an embodiment a first pressure of about 1.5 mbar is used for about 2 mins, the vacuum is then turned off for about 5 mins and the pressure increases, then the vacuum is applied again for about 2 mins with a resulting decrease in pressure, and the source is then turned off again for about 5 mins. This cycle of about 2 mins vacuum on and about 5 mins vacuum off, may be repeated between 5 and 50 times, between 10 and 50 times, between 20 and 50 times, for example 30, 32, 34, 36, 38, or 40 times.

Preferably the pressure is set by applying a vacuum to the sample. When the vacuum is applied the pressure is reduced.

The vacuum drying process may be performed for about 30 minutes to about 24 hours. The vacuum drying process may be performed for about 1 hour to about 18 hours, about 2 hour to 12 hours, about 2 hours to about 6 hours or about 3 hours to about 5 hours. Preferably, the vacuum drying process is performed for about 4 to 5 hours.

The vacuum drying process may comprise step of using a condenser. The purpose of the condenser is to attract the vapour away from the cornea. Thus, the vacuum drying process may comprise a condenser temperature of about −100° C. to about −30° C., about −90° C. to about −40° C. or about −100° C. to about −30° C. Preferably, the vacuum drying process may comprise a condenser temperature of about −55° C.

Thus, in one embodiment, the vacuum drying process may be performed for about 4 hours, at about 25° C., at about 990 μbar and comprise a condenser temperature of about −55° C.

In a further embodiment, the vacuum drying process may be performed for about 4 hours, at about 25° C., wherein the corneal tissue is initially subjected to a pressure of about 1.5 mbar for 2 mins, the vacuum pump is then turned off for 5 mins and the pressure on the tissue increases, the pump is then turned on for a further 2 mins and off for 5 mins, this cycle of 2 mins on and 5 mins off is repeated for about 36 cycles; a condenser temperature below 0° C., for example about −55° C., is used. In this method the pressure may not return to the original about 1.5 mbar in subsequent 2 minute parts of the cycle after the vacuum pump has been turned off for 5 minutes, however, the pressure will decrease below that observed in the 5 minute vacuum off period in the 2 minute vacuum on period.

The method of the second aspect of the invention may be used to produce an isolated dehydrated corneal tissue according to the first aspect of the invention.

According to a third aspect, the invention provides dehydrated corneal tissue produced by the method of the invention.

According a fourth aspect, the invention provides isolated dehydrated corneal tissue according to any aspect of the invention for use in therapy.

In a fifth aspect, the invention provides isolated dehydrated corneal tissue according to any aspect of the invention for use in treating or preventing corneal blindness in a subject.

In a sixth aspect, the invention provides isolated dehydrated corneal tissue according to any aspect of the invention for use in treating a damaged, diseased or infected eye in a subject. The dehydrated corneal tissue may be used to treat keratoconus, corneal melts, corneal ulcers and/or corneal perforation, and/or in tectonic support or anterior lamellar keratoplasty applications.

The invention may not be used to treat damaged, diseased or infected eyes, or endothelial dystrophies. The invention may be used to provide partial thickness tissue to treat conditions of the epithelium or stroma.

In a seventh aspect, the invention provides a method of treating a subject suffering from corneal blindness, the method comprising: obtaining isolated dehydrated corneal tissue according to the invention; rehydrating the corneal tissue and transplanting the rehydrated corneal tissue into a subject in need thereof.

In an eighth aspect, the invention provides a method of treating a subject with a damaged, diseased or infected eye, the method comprising: obtaining isolated dehydrated corneal tissue according to the invention; rehydrating the corneal tissue and transplanting the rehydrated corneal tissue into a subject in need thereof. The subject may have keratoconus.

The donor can refer to the individual from which the cornea has been obtained. Preferably, the donor is a deceased individual. The donor according to any aspect of the invention may be a vertebrate, mammal or domestic mammal. The donor may be a human, pig, cat, dog, horse, sheep or cow. Preferably, the donor is a human.

The subject according to any aspect of the invention may be a vertebrate, mammal or domestic mammal. The subject may be a human, pig, cat, dog, horse, sheep or cow. Preferably, the subject is a human.

Prior to use dehydrated corneal tissue according to the invention may be rehydrated. The tissue may be rehydrated using a rehydration solution. The rehydration solution may be a biocompatible water-based solution, such as a saline. The rehydration solution may comprise dextran. Thus, the rehydration solution may be saline (preferably 0.9% w/v NaCl) and 5% w/v dextran in water.

According to a ninth aspect, the invention provides a kit comprising dehydrated corneal tissue according to the invention, and a rehydration solution.

The terms “substantially all” or “substantially the entire” as used herein can be at least or greater than about 30%, 40%, 50%, 60%, 70%, 80%, 85%, 90%, 95%, 98%, 99%, or 100% of the diameter and/or thickness of the feature referred to herein, for example, the diameter and/or thickness of the Bowman's membrane or the Descemet's membrane.

The term “some” as used herein can be less than about 100%, 90%, 80%, 70%, 60%, 50%, 40%, 30%, 20%, 10%, 5%, 2% or 1% of the diameter and/or thickness of the feature referred to herein, for example, the diameter and/or thickness of the limbus.

All of the features described herein (including any accompanying claims, abstract and drawings), and/or all of the steps of any method or process so disclosed, may be combined with any of the above aspects in any combination, unless stated otherwise with reference to a specific combinations, for example, combinations where at least some of such features and/or steps are mutually exclusive.

For a better understanding of the invention, and to show how embodiments of the same may be carried into effect, reference will now be made, by way of example, to the accompanying drawings, in which:

FIG. 1 shows the structure of an eye. FIG. 1A shows the location of the cornea, limbus and sclera within the eye. FIG. 1B shows the structure of the cornea in cross section including the epithelial layer (the epithelium), Bowman's membrane, the stroma, Descemet's membrane and the endothelial layer (the endothelium).

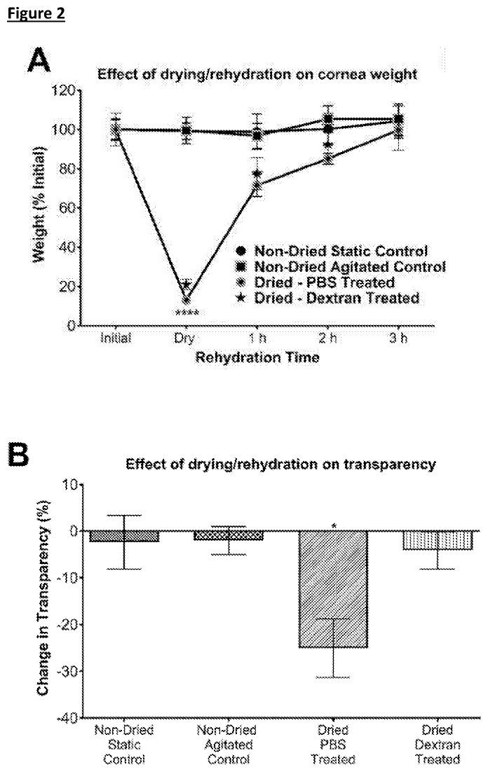

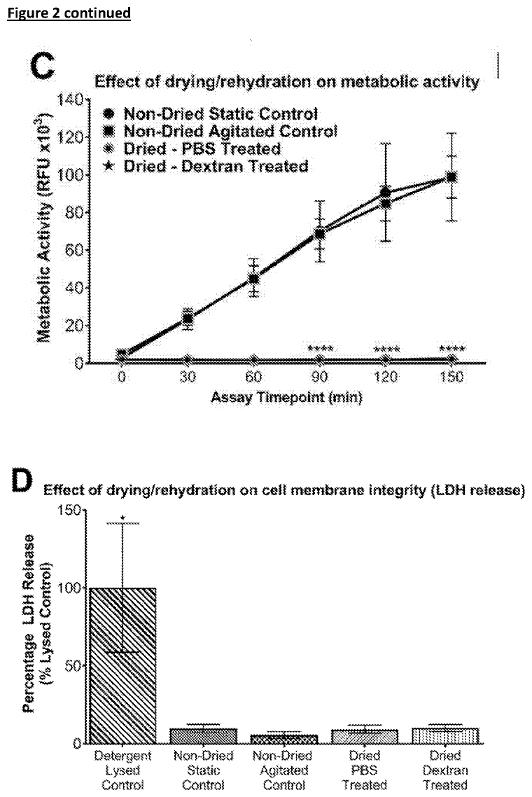

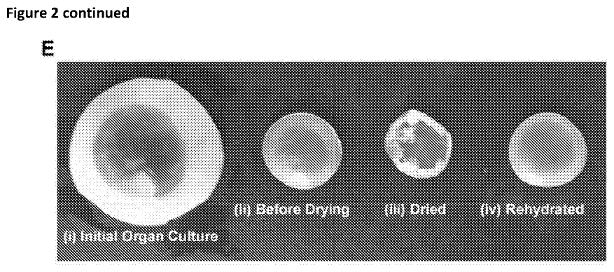

FIG. 2 shows the effect of low temperature vacuum evaporation on weight, transparency and metabolic activity of human corneal buttons. Human corneal buttons were dried after agitation in PBS, 5% dextran and compared to non-dried controls that had remained static or were agitated. Dried corneal buttons were rehydrated in PBS. FIG. 2A shows the change in weight of corneal buttons upon drying and rehydration for 3 hr. Data displayed as % of initial weight. FIG. 2B shows the change in transparency of corneal buttons after drying and rehydration. Data shown as percentage change in transparency from initial, represented as mean±SEM (n=5). FIG. 2C illustrates the metabolic activity of cells within corneal buttons measured over time after drying and rehydration. FIG. 2D shows LDH released from sample after rehydration versus as a percentage of the SDS-lysed control. Data for FIGS. 2A-2D are represented by mean±SEM (n=5). Statistical significance vs. non-dried static: *p≤0.05, **p≤0.01, ***p≤0.001, ****p≤0.0001. FIG. 2E shows images of (i) Organ culture cornea prior to drying, (ii) 8.5 mm corneal button before drying, (iii) corneal button after drying, (iv) corneal button after drying and rehydration;

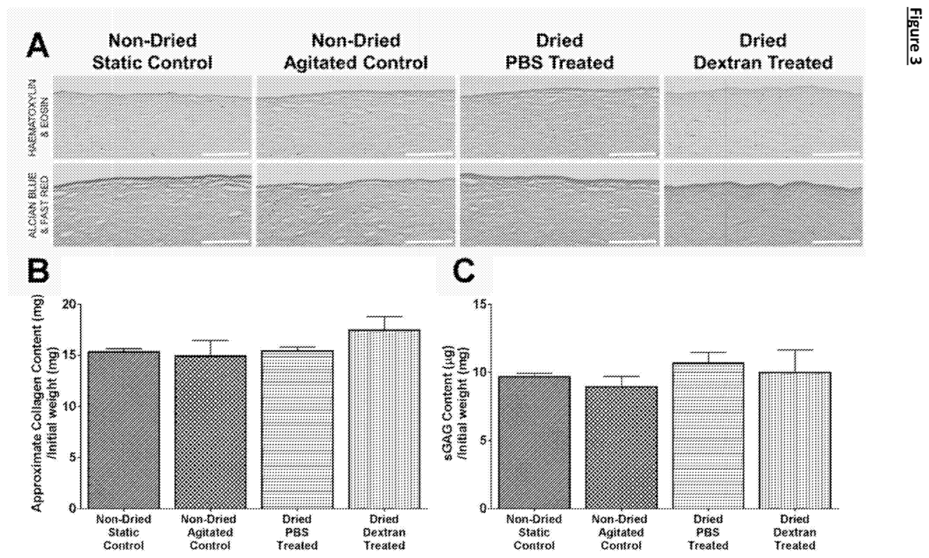

FIG. 3 shows the effect of low temperature vacuum evaporation on structure of human corneal buttons. Human corneal buttons were dried after agitation in PBS or in 5% dextran and compared to non-dried controls that had remained static or were agitated. Dried corneal buttons were rehydrated in PBS. FIG. 3A shows representative images of haematoxylin and eosin staining (upper row) and alcian blue and fast red staining (lower row) of sections of treated corneal buttons. Scale bar=200 μm. FIG. 3B shows the approximate collagen content of corneal buttons with and without drying and rehydration measured by hydroxyproline assay. FIG. 3C shows the approximate sGAG content of corneal buttons with and without drying and rehydration measured by DMMB assay. Data represented by mean±SEM (n=5);

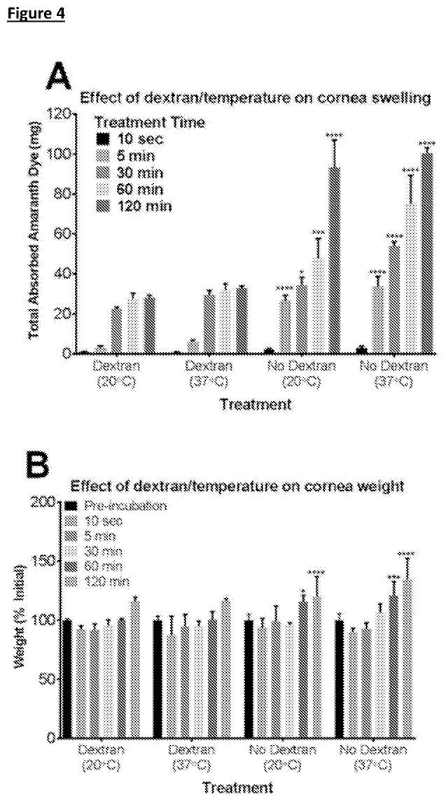

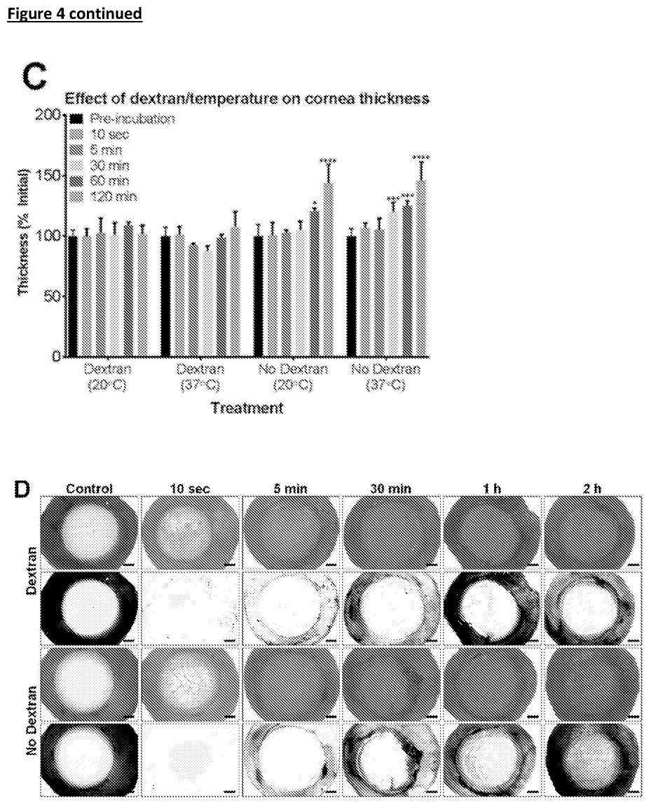

FIG. 4 shows the effect of dextran on corneal swelling during washing and agitation before drying. Whole corneas with sclera were agitated with 100 mM Amaranth Red dye with and without 5% dextran, at 20° C. or 37° C., for 10 s, 5 min, 30 min, 1 h or 2 h. FIG. 4A shows the total absorbed Amaranth dye during treatment. FIG. 4B shows the change in weight of corneas after treatment, represented as percentage of initial weight. FIG. 4C shows the change in thickness of corneas after treatment, represented as a percentage of initial thickness. Data represented mean±SEM (n=3). Statistical significance vs. same timepoint in dextran at 20° C.: *p≤0.05, ***p≤0.001, ****p≤0.0001. FIG. 4D shows stereo microscope images of Amaranth red dyed corneas, incubated at 20° C. (upper rows). Same images manipulated to better show levels of swelling (lower rows). Scale bar=2 mm;

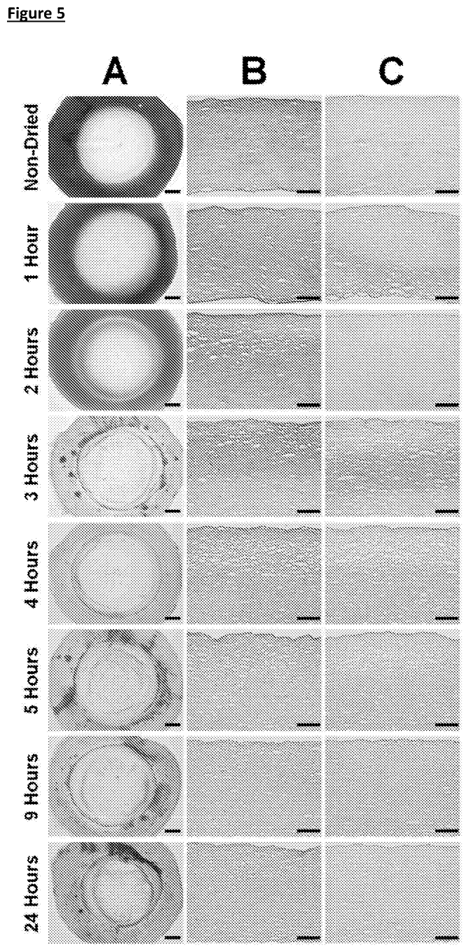

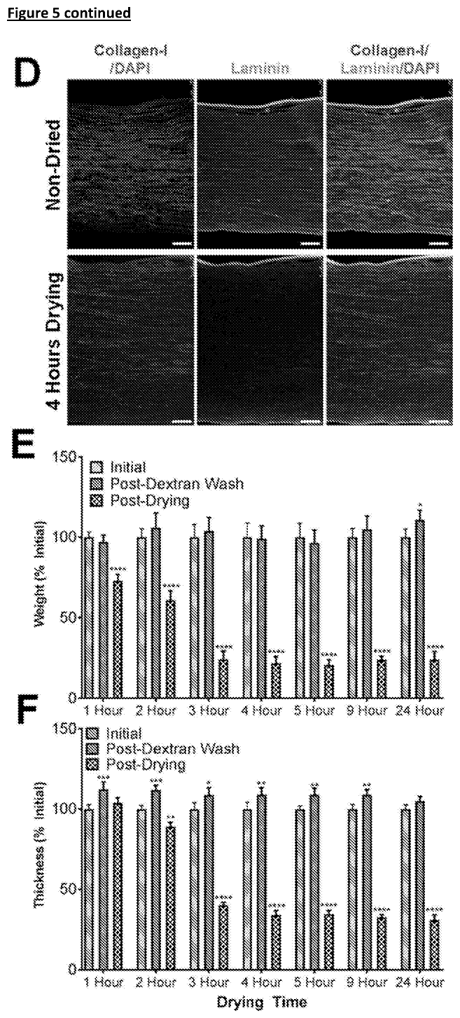

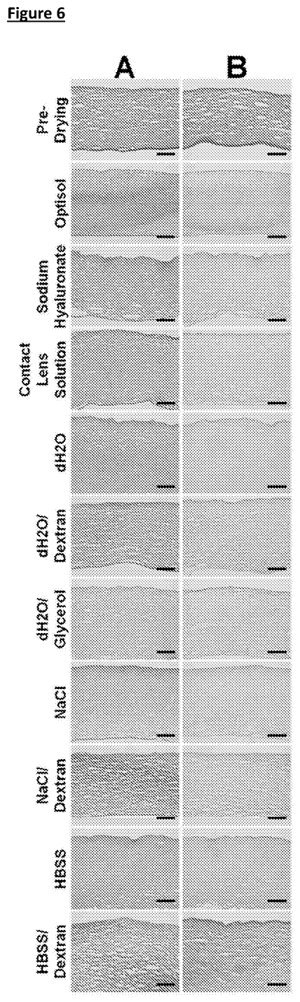

FIG. 5 concerns the optimisation of the low temperature vacuum evaporation drying time. Whole human corneas were agitated in 5% dextran in NaCl for 60 min before drying. Drying timepoints of 1 h, 2 h, 3 h, 4 h, 5 h, 9 h and 24 h were analysed with separate corneas. FIG. 5A shows stereo microscope images of corneas at different drying points. Scale bar=2 mm. FIG. 5B shows representative images of haematoxylin and eosin staining and FIG. 5C shows Alcian Blue and Fast Red staining. Scale bar=200 μm. FIG. 5D shows representative fluorescent immunohistochemistry images of sections of human corneas either non-dried or dried for 4 hours. Scale bar=100 μm. FIG. 5E shows the change in weight of corneas upon drying. Data displayed as % of initial weight. FIG. 5F shows the change in thickness of corneas upon drying. Data displayed as % of initial thickness. Data for FIGS. 5E and 5F represent mean±SEM (n=5). Statistical significance vs. initial: *p≤0.05, **p≤0.01, ***p≤0.001, ****p≤0.0001;

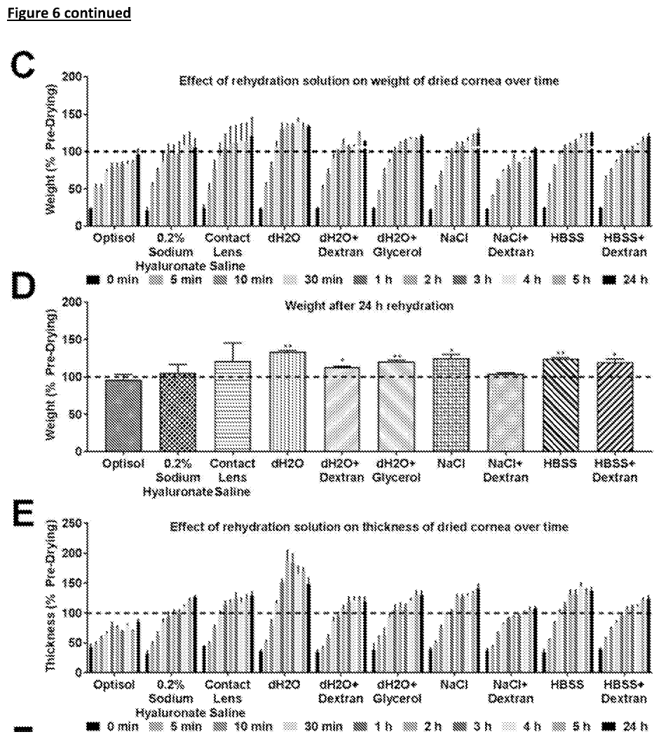

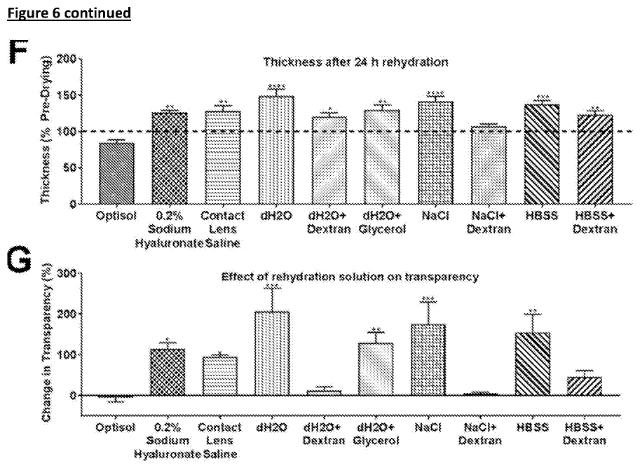

FIG. 6 shows the effect of different rehydration solutions on final properties of dried corneas. Whole human corneas were agitated in 5% dextran for 60 min before drying for 4 hours. Rehydration was performed with different solutions for up to 24 h. FIG. 6A shows representative images of haematoxylin and eosin staining and FIG. 6B shows Alcian Blue and Fast Red staining after rehydration for 24 h. Scale bar=200 μm. FIG. 6C shows the change in dried cornea weight over time in different rehydration solutions. Data displayed as pre-drying weight. FIG. 6D shows the dried cornea weight after 24 h rehydration. Data displayed as % pre-drying weight. FIG. 6E shows the change in dried cornea thickness over time in different rehydration solutions. Data displayed as % pre-drying thickness. FIG. 6F shows dried cornea thickness after 24 h rehydration. Data displayed as % pre-drying thickness. FIG. 6G shows the effect of rehydration solution on dried cornea transparency. Data is shown as percentage change in transparency from initial transparency. Data for FIGS. 6C-6G represent mean±SEM (n=3). Statistical significance vs. pre-drying: *p≤0.05, **p≤0.01, ***p≤0.001, ****p≤0.0001;

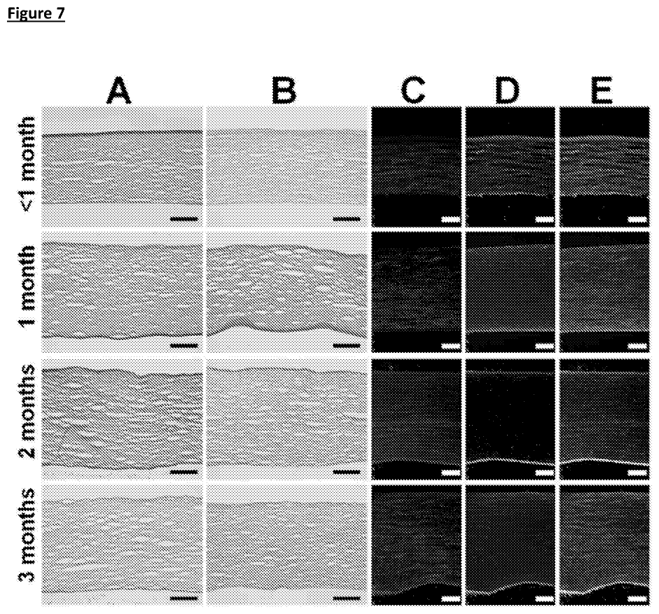

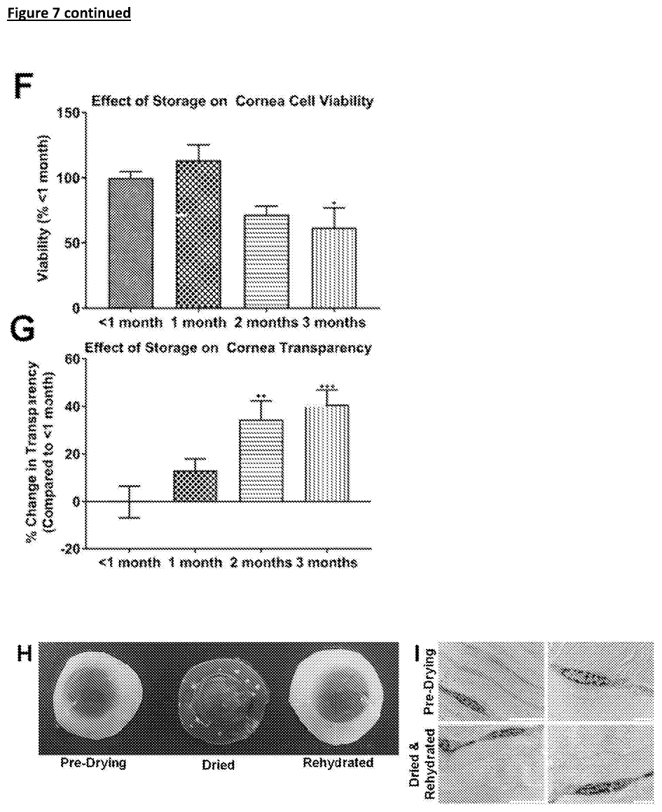

FIG. 7 shows the effect of prolonged storage prior to drying, and final structure of dried corneas. Whole corneas were stored in Optisol for up to 3 months before analysis. FIG. 7A is representative images of haematoxylin and eosin staining and FIG. 7B shows Alcian Blue and Fast Red staining after storage. Scale bar=200 μm. FIG. 7C shows representative images of collagen-I fluorescent immunohistochemistry after storage. FIG. 7D shows representative images of laminin fluorescent immunohistochemistry after storage. FIG. 7E shows merged images of collagen-I, laminin and DAPI. In FIGS. 7D-7E) the scale bar=100 μm. FIG. 7F shows the effect of storage prior to drying on cornea cell viability prior to drying. The data is represented as percentage of stored for <1 month. FIG. 7G shows the effect of storage prior to drying on cornea transparency. Data represented by change in transparency compared to corneas stored for <1 month. Data for FIG. 7G and FIG. 7H are represented by mean±SEM (n=4). Statistical significance vs. <1 month: *p≤0.05, **p≤0.01, ***p≤0.001. FIG. 7H shows macro-images of human corneas pre-drying, dried and rehydrated. FIG. 7I shows representative TEM images of cornea structure pre-drying and after drying and rehydration using optimized protocol. Scale bars: first column=5000 nm, second column=2000 nm; and

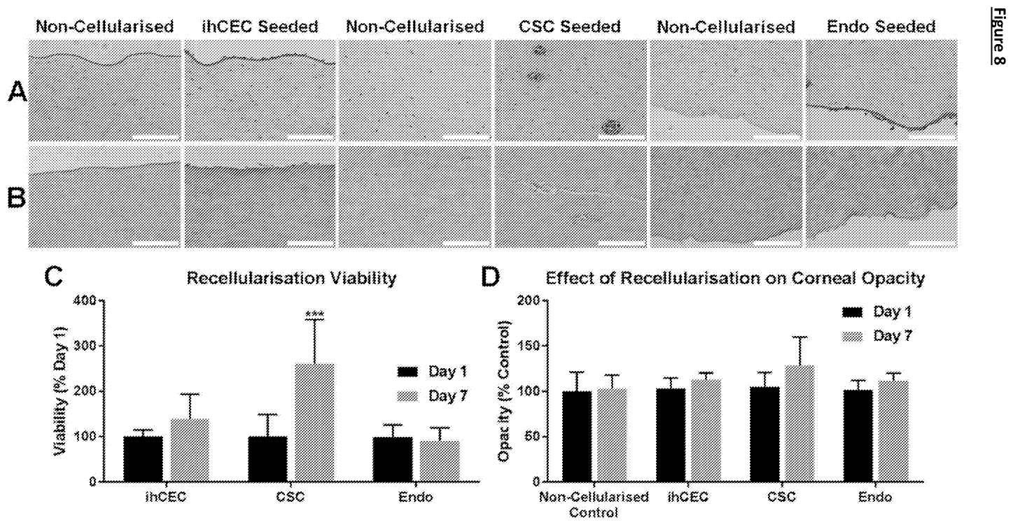

FIG. 8 shows the recellularisation of dried and rehydrated human corneas. Corneas were dried and rehydrated using the optimized protocol. Corneal buttons of 8 mm were recellularised with ihCEC, CSC or B4G12 corneal endothelial cells (Endo) for 7 days. FIG. 8A shows representative images of haematoxylin and eosin staining and FIG. 8B shows Alcian Blue and Fast Red staining after recellularisation. Scale bar=200 μm. FIG. 8C shows the relative proliferation and viability of cells grown on/in dried corneas. Data represented as % day 1 viability. FIG. 8D shows the effect of recellularisation on corneal opacity. Data represented as % non-cellularised control. Data for FIGS. 8C and 8D are represented by mean±SEM (n=5). Statistical significance vs. day 1: ***p≤0.001.

### EXAMPLES

Methods

Human Tissue

Anonymised human corneas were obtained from SightLife (now CorneaGen, WA, USA) under a materials transfer agreement. All work was performed in a laboratory under a research license from the Human Tissue Authority. Informed consent was obtained from donors/relative prior to collection.

Preparation and Drying of Corneas

Human corneas were delivered in Optisol™ (Bausch+Lomb, NJ, USA) and stored at 4° C. until use. Corneal buttons were prepared by punching out the middle of a cornea using an 8.5 mm trephine. Whole corneas with sclera were processed for drying as received from the eye bank with no further trimming. Corneal samples were transferred aseptically to a scintillation vial containing the sterile agitation media of either: Phosphate Buffered Saline (PBS) or 5% (w/v) dextran 70 (Sigma-Aldrich, Dorset, UK) in 0.9% NaCl. Scintillation vials containing corneas were agitated on a rotator (Grant PTR-60) at 60 rpm for up to 120 minutes, at either room temperature or 37° C. depending on experiment. Post-agitation, corneal samples were aseptically transferred to 10 mL glass vials (Schott via Adelphi Healthcare Packaging, West Sussex, UK) and a bromobutyl lyophilisation stopper (West Pharmaceutical Services via Adelphi) placed loosely on each vial. Vials were transferred to a Alpha 1-4 LSC, Advanced Freeze Dryer, Christ Osterode and dried at a shelf temperature of 25° C., pressure of 1.5 mbar, condenser temp −55° C. to −80° C. for different lengths of time depending on experiments. Once dried vial stoppers were pushed down and aluminium flip-up, tear-off crimp seal applied (West via Adelphi). Rehydration of corneal buttons or whole dried corneas was performed by injecting 3 mL sterile rehydration solution into the vial.

Unless otherwise stated, the standard, optimised procedure for drying the corneas was agitation in 5% (w/v) dextran in NaCl at room temperature for 60 minutes, drying for 4 hours and rehydration in 5% (w/v) dextran in NaCl.

Measurement of Corneal Weight and Thickness

Corneas weights were taken using an Ohaus Adventurer Balance. Corneas were place on pre-weighed weighboats and the weight of weighboats subtracted. Cornea thickness was measured using digital callipers.

Transparency Measurement

Light transmittance through the corneas was measured using a CLARIOstar plate reader (BMG LABTECH, Buckinghamshire, UK) at 492 nm with 12 readings taken across each cornea and averaged.

Metabolic Activity Assay

Metabolic activity was measured using PrestoBlue™ Cell Viability Reagent (Invitrogen, ThermoFisher, UK). Samples were placed in a 24-well plate and covered in 10% (v/v) Presto Blue reagent in Hank's Balanced Salt Solution (HBSS, Gibco, ThermoFisher). The plate was immediately transferred to a CLARIOstar plate reader pre-set at 37° C. and fluorescence readings at excitation 560 nm/emission 590 nm were taken every 30 minutes for 150 min.

Lactate Dehydrogenase (LDH) Release Assay

The Pierce lactate dehydrogenase (LDH) assay kit was used to quantify levels of LDH released into the rehydration media of dried corneas, to estimate levels of cell membrane lysis. The assay was performed according to the manufacturer's protocol. Briefly, 50 μL of rehydration media and 50 μL of reaction mix were transferred to a 96-well plate and incubated at room temperature for 30 min. The optical absorbance was read on the plate reader at 490 nm with background correction at 690 nm. The maximum levels of LDH that could be released from a cornea was assessed using non-dried corneas that had been agitated in 1% (v/v) sodium dodecyl sulphate detergent at 37° C. for 24 hours.

Histology

Samples were fixed in 4% paraformaldehyde overnight, before washing and storage in PBS. Samples were prepared for sectioning in a Tissue Processor (Leica TP1020) through a series of graded ethanol solutions, then paraffin embedded. Sections (7 μm) were cut using a Leica 2245 microtome and transferred to adherent glass slides (SuperFrost Plus, ThermoScientific). Samples were de-paraffinised in xylene and rehydrated in a series a graded ethanol solutions. Regressive haematoxylin and eosin staining was performed using Harris Haematoxylin and 1% eosin. Alcian blue and fast red staining was performed to visualise acid mucosubstances and red cell nuclei. Slides were mounted in DPX after staining and imaging was performed on a Leica DM1000 upright microscope and MC170 Camera.

Hydroxyproline Assay

Hydroxyproline assays were performed as described previously (Edwards and O'Brien 1980) to estimate the levels of collagen within the corneas. Corneal buttons were digested in a 0.1 mg/mL papain solution in 0.2 M sodium phosphate buffer containing 8 mg/mL sodium acetate, 4 mg/mL ethylenediaminetetraacetic acid, and 0.8 mg/mL L-cysteine hydrochloride agitated at 65° C. overnight. Briefly, acid hydrolysis of papain digested samples was achieved by heating samples with concentrated hydrochloric acid to 120° C. for 5 hours. Subsequently, samples were dried at 80° C. until only residue remained, which was dissolved in 0.2 M sodium phosphate buffer. Samples were transferred in triplicate to a 96-well plate, an equal volume of 70 mM chloramine T solution was added and incubated at room temperature for 20 minutes. Subsequently, an equal volume of 1.16 M dimethylaminobenzaldehyde solution was added and samples incubated at 60° C. for 30 minutes. Colour change was assessed by absorbance at 540 nm. Hydroxyproline concentration was calculated using a standard curve. Collagen concentration was estimated using a conversion factor of 7.6. Collagen readings were corrected for the original weight of the corneal button.

Sulphated Glycosaminoglycans (sGAG) Assay

Corneal buttons were digested in papain as described above. The Blyscan™ 1,9 dimethyl methylene blue (DMMB) assay (Biocolor Ltd., Belfast, UK) assay was performed on samples according to manufacturer's instructions. Briefly, 200 μL of papain digest was added to 1 mL DMMB dye solution and agitated for 30 minutes, before centrifugation at 10,000×g for 10 minutes. The pellet was dissolved in 0.5 mL dissociation reagent and 200 μL transferred to each well of a 96-well plate. Absorbance was measured at 656 nm. sGAG concentration was determined using a standard curve. sGAG readings were corrected for original weight of the corneal button.

Amaranth Dye Absorption Assay

Amaranth Red dye was used to assess swelling of corneas before drying during pre-treatment with and without dextran at different times and temperatures. Solutions of 100 mM Amaranth Red (Sigma-Aldrich) in 0.9% NaCl and 100 mM Amaranth Red with 5% (w/v) dextran 70 in 0.9% NaCl were prepared. Whole corneas with sclera were place in the Amaranth Red solutions. Samples were incubated at either room temperature (20° C.) or 37° C. on a rotator set at 60 rpm. At required timepoints samples were removed from rotator, imaged using a Leica S6 D stereo microscope and with MC170 Camera, weighed, thickness measured and the Amaranth dye washed back out into 3 mL 0.9% NaCl on a roller. Total amount of Amaranth Dye was washed out was measured by reading absorbance at 520 nm and comparing to an Amaranth Dye standard curve.

Images of Amaranth Red dyed corneas were analysed using image J version 1.52a. Images were split into separate RGB channels, and the red channel image adjusted for brightness and contrast identically in all images.

Fluorescent Immunohistochemistry

Corneas were paraffin-embedded and sectioned as for histology. Samples were deparaffinised in xylene and rehydrated in a series of graded ethanol solutions. Antigen retrieval was performed in a pH 6.0 sodium citrate buffer (Vector) at 95° C. for 60 min. Sections were permeabilised in 0.1% (v/v) Triton-X100 for 10 minutes and subsequently washed three times for 5 minutes in PBS. Non-specific protein binding was blocked using a solution of PBS with 1% bovine serum albumin (BSA), 0.3 M glycine and 3% (v/v) donkey serum, for 1 hour at RT. Primary antibodies were diluted in PBS containing 1% BSA and 0.3 M glycine as follows: polyclonal mouse anti-Collagen-I (Sigma-Aldrich, dilution 1:200) and polyclonal rabbit anti-laminin (Millipore, dilution 1:100) Sections were incubated with the primary antibodies for 1 hour at room temperature before washing three times in PBS. Either donkey anti-mouse Alexa-Fluor 594 or donkey anti-rabbit Alexa Fluor 488 secondary antibodies (Life Technologies, dilution 1:300) were applied to the samples at room temperature for 1 hour. Samples were rinsed in PBS three times and counterstained with 4′,6-diamidino-2-phenylindole (DAPI; 1:200,000, Life Technologies). Samples were mounted in fluorescent mounting medium (Dako, UK) and imaged using a Leica DMIL LED inverted microscope with a Leica DFC camera.

Rehydration

Rehydration was performed by immersing corneas in 5 mL of various rehydration solutions: Optisol (Bausch+Lomb), 0.2% sodium hyaluronate (HYLO-FORTE Eyedrops, Scope, UK), contact lens saline (Tesco, UK), distilled water (dH₂O), dH₂O with 5% (w/v) dextran, dH2O with 10% (v/v) glycerol, 0.9% NaCl, 0.9% NaCl with 5% (w/v) dextran, Hank's Balanced Salt Solution (HBSS, Gibco, ThermoFisher, UK), and HBSS with 5% (w/v) dextran.

Transmission Electron Microscopy

Samples were fixed in 3% glutaraldehyde in sodium cacodylate buffer solution (0.2 M, pH 7.2) for 24 h at 4° C. Corneas were post-fixed with 1% osmium tetroxide in sodium cacodylate buffer solution for 2 h at room temperature, followed by washing in sodium cacodylate buffer (0.1 M, pH 7.2), serial dehydrations in ethanol, and washing in propylene oxide (TAAB Laboratories Equipment Ltd). The corneas were embedded in araldite resin (TAAB Laboratories Equipment Ltd) and sectioned (Leica EM UC6; Leica Biosystems). Ultrathin sections of 90 nm were contrasted with uranyl acetate and lead citrate and observed on a transmission electron microscope (TEM, FEI Tecnai Biotwin T12), operating at 100 kV. Images were taken using a SIS Megaview digital camera (Olympus).

Recellularisation

Preparation of Dried Corneas for Recellularisation

Dried whole corneas were rehydrated in sterile NaCl with 5% (w/v) dextran for one hour. A sterile trephine was used to cut out a central corneal button of 8.5 mm diameter. Prior to CSC injection seeding, corneas were swollen in dH2O for 1 hour.

Immortalised Human Corneal Epithelial Cells (ihCEC)

SV40-immortalised human corneal epithelial cells (ihCEC) [Araki-Sasaki, K., et al., Invest Ophthalmol Vis Sci, 1995. 36(3): p. 614-21] were cultured in supplemented basal epithelial cell medium EpiLife® containing 5 mL human keratinocyte growth supplement and 1% antibiotic-antimycotic (AbAm, Sigma-Aldrich). ihCEC were seeded on the basement membrane side at 1×10⁶ cells per corneal button, and cultured in 3 mL of supplemented EpiLife containing 5% (w/v) dextran.

Corneal Stromal Cells (CSCs)

Corneal stromal cells were extracted from human corneoscleral rims by enzymatic means and cultured as described previously in Stem Cell Medium (SCM) consisting of DMEM/F12 with Glutamax supplemented with 20% (v/v) knock-out serum replacement (KSR), 1% (v/v) non-essential amino acids, 4 ng/mL bFGF, 5 ng/mL hLIF (New England Biolabs, Hertfordshire, UK) and 1% AbAm [Sidney, L. E., et al. Cytotherapy, 2015. 17(12): p. 1706-1722]. For recellularisation, CSC were re-suspended in 250 μL SCM containing 5% (w/v) dextran, and injected through a 20 gauge hypodermic needle into the stroma of swollen corneas through the endothelial side. CSC injected into corneas were then cultured in SCM containing 5% (w/v) dextran.

B4G12 Corneal Endothelial Cells (Endo Cells)

The B4G12 corneal endothelial cell line was cultured in Endothelial Serum-Free Growth Medium (Endo-SFM, Gibco, ThermoFisher) supplemented with 10 ng/mL basic fibroblast growth factor (ThermoFisher) and 1% AbAm. Endo cells were seeded on the Descemet's side at 1×10⁶ cells per corneal button, and cultured in 3 mL of supplemented Endo SFM containing 5% (w/v) dextran.

### Example 1

### The Effect of Drying on Corneal Characteristics

The data presented herein demonstrates that the dried weight of corneas is approximately 20% of initial weight, regardless of the agitation media used.

Rehydration of corneas dried according the invention was shown to return the corneas back to their original weight within 3 hours (see FIG. 2A). Pre-treatment in had a significant negative effect on transparency of the corneas (see FIG. 2B).

Non-dried corneas are shown to retain significant metabolic activity in the cells (cell viability), and drying is shown to lead to a loss of this metabolic activity. When pre-treated with PBS or dextran metabolic activity is completely lost (cells are non-viable).

Measuring the amount of LDH released into the rehydration media crudely measures the number of lysed cells and therefore the level of cell membrane bursting. No difference was observed in dried versus the non-dried corneas, and when corneas were treated with an SDS lysis agent, levels of LDH released were significantly higher than in any other group (see FIG. 2D).

Corneal buttons look very similar after drying and rehydration to how they look pre-dried (see FIG. 2E).

### Example 2

### Corneal Structure

No apparent change in corneal structure was caused by drying and rehydration (see FIG. 3A). No difference in structure was observed when different pre-treatments were used. No loss of collagen or sGAG content was observed by drying and rehydration (see FIGS. 3B and 3C).

### Example 3

### The Effect of Dextran on Corneal Structure

Dextran is shown to prevent further absorption of dye after 30 minutes, therefore demonstrating its ability to prevent swelling of the cornea to let in more dye (see FIG. 4A). Without dextran present the cornea was observed to swell to beyond its original weight and thickness (see FIGS. 4B and 4C). The difference in the levels of dye penetrating the corneas can be seen in the images (see FIG. 4D).

The presence of dextran meant that the temperature of agitation had no effect.

### Example 4

### The Effect of Different Drying Times on Corneal Structure

Optimal drying time is shown in the data presented herein to be 4 hours. After 4 hours the weight/thickness of the cornea does not reduce any further. Drying for 24 hours is shown not to change the corneal structure (see FIGS. 5A to 5C, 5E and 5F). Dried corneas are observed to retain the basement/Bowman's membrane and Descemet's membranes (showed by laminin staining) but there are no epithelial or endothelial cells (see FIG. 5D).

### Example 5

### The Effect of Corneal Rehydration Solutions on Corneal Structure

Rehydration of a corneal structure with a solution containing NaCl and dextran produced a rehydrated tissue closest to initial weight and thickness and did not affect structure or transparency (see FIG. 6). Water alone was shown to cause significant swelling and loss in transparency on rehydration.

### Example 6

### The Effect of Storage Prior to Drying

Storage of corneas in Optisol™ for longer than one month prior to drying resulted in a loss of epithelial cells and endothelial cells, but not a loss of basement membrane or Descemet's (see FIGS. 7A to 7E). Cell viability is also lost over storage time (see FIG. 7F), but metabolic activity is maintained for 3 months. A loss of transparency over storage time was also observed (see FIG. 7G). Macro images show that rehydrated corneas look similar to those before drying. Pink staining of pre-drying is due to Optisol™ storage (see FIG. 7H). TEM images show the microstructure of collagen fibrils are not affected by drying and rehydration (See FIG. 7I). Stromal cells are encapsulated within the collagen fibrils.

### Example 7

### Analysis of Rehydrated Corneas

Dried and rehydrated corneas are biocompatible with all three major cellular types of the cornea. Epithelial cells attach to the basement membrane (see FIGS. 8A and 8B). Endothelial cells attached to the Descemet's membrane. CSCs fill pockets within the corneal stroma and proliferate within the cornea. Transparency is not significantly affected by recellularisation (see FIG. 8D).

1. An isolated dehydrated corneal tissue, comprising a full thickness corneal stroma, and substantially all, or all, of the Bowman's membrane, wherein the stroma contains cellular material.

2. The isolated dehydrated corneal tissue of claim 1 wherein the tissue comprises some or substantially all, or all, of the Descemet's membrane.

3. The isolated dehydrated corneal tissue of claim 1 or claim 2 wherein the tissue does not comprise an epithelium or an endothelium.

4. The isolated dehydrated corneal tissue of any of claims 1 to 3 wherein the tissue consists of an entire corneal stroma, and substantially all or all of the Bowman's membrane.

5. The isolated dehydrated corneal tissue of any of claims 1 to 3 wherein the tissue consists of an entire corneal stroma, substantially all, or all, of the Bowman's membrane and some or substantially all, or all, of the Descemet's membrane.

6. The isolated dehydrated corneal tissue of any preceding claim wherein in the stroma of the dehydrated corneal tissue at least some of the corneal keratocytes are retained.

7. The isolated dehydrated corneal tissue of any preceding claim wherein the tissue does not comprise any viable/living cells or live cellular layers from the donor.

8. The isolated dehydrated corneal tissue of any preceding claim wherein the tissue is not prepared by freeze-drying.

9. The isolated dehydrated corneal tissue of any preceding claim wherein the tissue has not been lathed.

10. A method of producing dehydrated corneal tissue, the method comprising:
a) providing corneal tissue that has been obtained from a donor;
b) suspending the corneal tissue in a solution comprising an osmotic agent;
c) agitating the solution in which the corneal tissue is suspended in order to remove the endothelial cells and epithelial cells, whilst the stroma retains cellular material; and
d) vacuum drying the corneal tissue from c) in order to produce a dehydrated corneal tissue.

11. The method of claim 10 wherein the corneal tissue provided in step (a) comprises substantially all, or all, of the epithelium; substantially all, or all, of the Bowman's membrane; substantially all, or all, of the stroma; substantially all, or all, of the Descemet's membrane; and substantially all, or all, of the endothelium.

12. The method of claim 9 or 10 wherein the corneal tissue after step (c) comprises substantially all, or all, of the Bowman's membrane; substantially all, or all, of the stroma; and substantially all, or all, of the Descemet's membrane.

13. The method of claim 12 wherein after step (c) the corneal tissue does not comprise the epithelial layer (the epithelium) and/or the endothelial layer (endothelium).

14. The method of any of claims 10 to 13 wherein the dehydrated corneal tissue produced contains at least some keratocytes in the stroma of the tissue.

15. The method of any of claims 10 to 14 wherein the corneal tissue after step (c) comprises all or substantially all of the Bowman's membrane and the stroma.

16. The method of claim 15 wherein after step (c) the corneal tissue further comprises all or substantially all of the Descemet's membrane.

17. The method of any of claims 10 to 16 wherein the corneal tissue after step (c) consists of all or substantially all of the Bowman's membrane and all or substantially all of the stroma.

18. The method of any of claims 10 to 16 wherein the corneal tissue after step (c) consists of all or substantially all of the Bowman's membrane, all or substantially all of the stroma, and all or substantially all of the Descemet's membrane.

19. The method of claim 10 wherein the Descemet's membrane and/or the Bowman's membrane of the donor are not intentionally removed from the corneal tissue.

20. The method of any of claims 10 to 19 wherein the corneal tissue comprises substantially the entire diameter of the donor's cornea.

21. The method of any of claims 10 to 20 wherein the stroma of the corneal tissue is of full thickness.

22. The method of any of claims 10 to 21 wherein the osmotic agent is a salt.

23. The method of any of claims 10 to 22 wherein the osmotic agent is a glucose polymer, such as dextran.

24. The method of any of claims 10 to 23 wherein the solution further comprises a drying protectant.

25. The method of any of claims 10 to 24 wherein the agitation in step (c) is intended to remove all or substantially all the cells in the epithelial layer and all the cells in the endothelial layer.

26. The method of any of claims 10 to 25 wherein the method does not include a step in which the tissue is lathed.

27. The method of any of claims 10 to 26 wherein the vacuum drying step is performed by vacuum evaporation.

28. An isolated dehydrated corneal tissue produced according to the method of any of claims 10 to 27.

29. An isolated dehydrated corneal tissue according to any of claims 1 to 9 or claim 28 for use in therapy.

30. An isolated dehydrated corneal tissue according to any of claims 1 to 9 or claim 28 for use in treating or preventing corneal blindness in a subject.

31. An isolated dehydrated corneal tissue according to any of claims 1 to 9 or claim 28 for use in treating a damaged, diseased or infected eye in a subject.

32. The dehydrated corneal tissue for the use of claim 31 wherein the tissue if for use in treating keratoconus, corneal melts, corneal ulcers and/or corneal perforation, and/or in tectonic support or anterior lamellar keratoplasty applications.

33. A method of treating a subject suffering from corneal blindness, the method comprising: obtaining isolated dehydrated corneal tissue according to any of claims 1 to 9 or claim 28; rehydrating the corneal tissue and transplanting the rehydrated corneal tissue into a subject in need thereof.

34. A method of treating a subject with a damaged, diseased or infected eye, the method comprising: obtaining isolated dehydrated corneal tissue according to any of claims 1 to 9 or claim 28; rehydrating the corneal tissue and transplanting the rehydrated corneal tissue into a subject in need thereof.

35. A kit comprising dehydrated corneal tissue according to any of claims 1 to 9 or claim 28, and a rehydration solution.
